(19) United States
(12) Patent Application Publication
(10) Pub. No.: US 2024/0127917 A1
(43) Pub. Date: Apr. 18, 2024

(54) METHOD AND SYSTEM FOR PROVIDING A DOCUMENT MODEL STRUCTURE FOR PRODUCING A MEDICAL FINDINGS REPORT

(71) Applicant: Siemens Healthcare GmbH, Erlangen (DE)

(72) Inventors: Christian TIETJEN, Fuerth (DE); Sven KOHLE, Erlangen (DE); Christoph SPEIER, Erlangen (DE)

(73) Assignee: Siemens Healthcare GmbH, Erlangen (DE)

(21) Appl. No.: 18/488,490

(22) Filed: Oct. 17, 2023

(30) Foreign Application Priority Data
Oct. 18, 2022 (DE) 102022211036.6

(51) Int. Cl. G16H 15/00; G16H 50/70

(52) U.S. Cl. CPC G16H 15/00; G16H 50/70

(57) ABSTRACT

A method for locating or identifying components or elements based on which a medical findings report can be produced for a patient to be assessed includes: providing a plurality of comparison datasets, each comparison dataset having at least one reference medical findings report; providing an analysis function, which is configured to ascertain, for a medical dataset of the patient being assessed, at least one reference dataset from the plurality of comparison datasets; ascertaining at least one reference dataset from the plurality of comparison datasets by applying the analysis function to the medical dataset and the comparison datasets; identifying at least one document model structure for the patient to be assessed based on the at least one reference medical findings report associated with the at least one reference dataset; and providing the at least one document model structure for further processing.

### CROSS-REFERENCE TO RELATED APPLICATION(S)

The present application claims priority under 35 U.S.C. § 119 to German Patent Application No. 10 2022 211 036.6, filed Oct. 18, 2022, the entire contents of which are incorporated herein by reference.

### FIELD

One or more example embodiments of the present invention relate to methods and systems for use in medical assessment. Methods and apparatuses according to embodiments of the present invention can be used in particular in producing medical findings reports. Embodiments of the present invention relate in particular to methods and systems for locating or identifying, in particular predetermined, components or elements, on the basis of which a medical findings report can be produced, which locating is performed in a manner appropriate to the particular patient to be assessed.

### BACKGROUND

In the assessment of a medical dataset, abnormalities are described in a medical findings report. In this process, abnormalities are marked or even measured in the medical dataset, and noted in the findings report.

The assessment is a complex activity, as is the production of medical findings reports. For example, radiologists must typically carry out several tasks in parallel when evaluating medical data and producing a findings report. First and foremost, they must analyze the key medical (image) data of the patient, and summarize their observations and impressions in a findings report. Furthermore, they must take account of additional information about the patient. For example, this information can originate from images from different modalities or measurement protocols, and can come from information in the medical record of the patient, from laboratory findings, earlier acquisitions, etc. The nature and number of the individual work packages in the assessment, and hence in the findings report to be produced, depend on numerous factors, for instance the available interdisciplinary data, the patient status, the assessment task, the health status, the existing suspected diagnoses, etc.

It is not always clear to the assessor how best to structure the findings report in a given case, or which further aspects should be considered. In addition, a diagnosis based on the existing data and additional information is not always conclusive, and possible differential diagnoses exist that have to be clarified in a further diagnostic process.

What are known as structured findings reports have been introduced in order to offer assistance to the assessors. These are based on structured, machine-readable (report) models or “template components”, which can be combined and completed step-by-step by the assessor in order to provide the final medical findings report. Ideally, a structured findings report is machine-readable, has a fixed structure and contains standardized elements, phrases and layouts. In addition, ready-made report models or modules can be used as components for the medical findings report. These can offer a case-specific structure and can contain recommended assessment steps.

Although structured findings reports can improve the quality and interoperability of medical reports, this can also increase the time and effort of the individual assessor in producing the findings report. Instead of dictating the medical findings relatively freely, the assessor must select the correct models or components and fill these in a defined manner. Furthermore, an assessor must usually choose from a large number of different models. Finding the correct model is often crucial to the entire process, because an incorrect model can take the assessment, and hence the entire diagnosis, along the wrong path. Selecting the correct models is again a difficult task, because this can depend on various factors such as the diagnostic task, the symptoms, the suspected diagnoses to be clarified, or the available data. In addition, there is rarely one single model for a medical findings report. Instead, different modules or sub-models must be combined in order to arrive at a comprehensive medical findings report.

### SUMMARY

At least one object of embodiments of the present invention is to provide methods and systems that assist an assessor or user in producing a medical findings report. In particular, it is an object of embodiments of the present invention to provide methods and systems that provide the user with document model structures for producing a medical findings report that are appropriate for the specific case and on the basis of which the user can produce a medical findings report in an expedient and efficient manner.

These and further objects are achieved by a method, a system, a non-transitory computer program product or a non-transitory computer-readable storage medium, respectively, as claimed in the main claim and the additional independent claims, and as described herein. The dependent claims and the following description also describe advantageous developments.

The manner in which one or more embodiments of the present invention achieve at least the object is described below with reference to the claimed systems and with reference to the claimed methods. Features, advantages or alternative embodiments/aspects mentioned in this connection can be assigned likewise to the other claimed subject matter, and vice versa. In other words, the object-based claims (which are directed at a system, for example) can also be developed by combining with the features described or claimed in connection with a method. The corresponding functional features of the method can also be embodied by corresponding object-related modules.

In addition, the manner in which one or more example embodiments of the present invention achieve the object is described also with reference to methods and systems for adapting trained functions. Here, features and alternative embodiments/aspects of data structures and/or functions in methods and apparatuses in the application of a trained function can be assigned to analogous data structures and/or functions in methods and systems for said adapting. Said analogous data structures may be identified in particular by the use of the prefix “training”. In addition, the trained functions used in methods and apparatuses may have been adapted and/or provided in particular by methods and systems for adapting trained functions.

According to one aspect, a computer-implemented method for providing a document model structure is provided. Said document model structure is suitable as the basis for producing a medical findings reports as part of an assessment of a patient to be assessed. The method comprises a plurality of steps. One step is directed at receiving in a computing facility a medical dataset of the patient to be assessed. A further step is directed at providing in the computing facility a plurality of comparison datasets, which differ from the medical dataset, wherein each comparison dataset has at least one reference medical findings report. A further step is directed at providing an analysis function which is designed to ascertain for a medical dataset at least one reference dataset from a plurality of comparison datasets. A further step is directed at ascertaining by the computing facility at least one reference dataset from the plurality of comparison datasets by applying the analysis function to the medical dataset and the comparison datasets. A further step is directed at identifying by the computing facility on the basis of the at least one reference medical findings report associated with the at least one reference dataset at least one document model structure for the patient to be assessed. A further step is directed at providing the identified at least one document model structure by the computing facility for the purpose of producing a medical findings report for the patient to be assessed.

A medical findings report can be the result of a diagnostic process that aims to determine the condition of a patient with regard to one or more clinical aspects on the basis of the medical data relevant thereto. A medical findings report can have a document. In particular, a medical findings report can have a structured document or structured document parts. In addition, a medical findings report can have an unstructured document or unstructured document parts. In addition, the medical findings report can be constructed from one or more template components, into which patient-specific information is entered as part of the assessment.

Data produced by imaging methods, for instance in particular X-ray images, magnetic resonance scans or ultrasound acquisitions, or histopathology images, can form part of the medical findings report and is used in particular for the documentation and transparency of the diagnostic process. Similarly, the medical findings report can contain non-image data.

In addition, data can also be derived from medical datasets via data processing. This can be measured values (for instance volumes, distances, contrast values, area ratios, etc.) extracted, in particular automatically, from image data, or measured values (trends, absolute values, etc.) extracted, in particular automatically, from non-image data. Again, a findings report can contain such data.

It is also the responsibility of a user to interpret or appraise the medical dataset or individual items of information in terms of symptoms. The results of the appraisal of the patient's data can also be included in the medical findings report, for example as the assessment text.

The medical data can comprise data and information available on the patient to be assessed. Said medical data can comprise all, or just some, of the data and information available on the patient to be assessed. The medical data can comprise both medical image data and non-image data. In this context, image data can relate to medical image data having two or three spatial dimensions. Furthermore, the image data can additionally have a time dimension. Medical image data is in particular image data that has been acquired with an imaging modality, and in particular can depict a part of the body of the patient. Imaging modalities can comprise here, for example, computed tomography devices, magnetic resonance devices, X-ray devices, ultrasound devices and the like. Image data acquired using these or similar modalities is also referred to as radiology image data. In addition, medical image data can comprise digitized histopathology images, which depict an appropriately prepared tissue section from the patient. The image data can further comprise longitudinal data, for instance in the form of time series or successive acquisitions that are spaced in time.

Non-image data can comprise, in particular longitudinal, data containing one or more medical values for the patient and/or elements from the medical history of the patient. This may be laboratory data, vital signs and/or other measured values or preliminary examinations relating to the patient. In addition, the non-image data can comprise patient-related demographic details, for instance relating to the age, gender, lifestyle habits, risk factors, etc.

The medical data can be retrieved from one or more storage facilities, which storage facilities may be incorporated in a medical information network. For example, the user can select an assessment task or a patient from a job list in a front-end computing facility. Based on the selection made for the assessment task or the patient, the patient data for the patient can be requested from the connected storage facilities (e.g. by the computing facility or the front-end computing facility). For example, an electronic identifier, for instance a patient ID or an access number, can be used for this purpose. The medical data can accordingly be received from one or more of the available storage facilities, in each of which is stored at least some of the medical data. For example, the storage facilities can be part of medical information systems such as hospital information systems and/or PACS systems and/or laboratory information systems, etc.

According to some embodiments/aspects of the present invention, the medical data can be very comprehensive and have diverse information about the health condition of the patient (according to other embodiments/aspects of the present invention, however, it can also be restricted to just one data category, for instance image data and then specifically radiology image data). It can be a task of the user as part of an assessment task, based on the medical data, to make a medical finding or medical diagnosis or medical conclusion and produce a medical findings report.

The comparison datasets can have substantially the same form as the medical dataset. Unlike the medical dataset, however, each of the comparison datasets already has available at least one medical findings report as a reference findings report. The comparison datasets (at least some) can be from different patients than the patient to be assessed. In addition, the comparison datasets can also have, in particular older, datasets of the patient to be assessed.

According to some examples, the comparison datasets can each have just one medical findings report. In other words, the comparison datasets can be understood as a collection or archive of existing findings reports.

In particular, the reference findings reports can have finalized findings reports. Finalized findings reports may have been signed and/or verified. For example, the findings reports may have been signed and/or verified by the user or one or more further users (who differ from the user). The reference findings reports can each be based on at least one document model structure or comprise at least one document model structure.

The analysis function can be regarded in particular as a computer program product which is designed to select from a plurality of comparison datasets one or more reference datasets that “fit” a medical dataset. The analysis function can have program constituents in the form of one or more instructions for the computing facility for ascertaining the reference datasets. For example, the analysis function can be provided by being held in a storage facility or being loaded into a main memory of the computing facility or by being made available generally for use.

The analysis function is designed to provide on the basis of input data (comprising at least one medical dataset and a plurality of comparison datasets) one or more reference datasets as output data. For the selection of suitable reference datasets, the analysis function can implement different methods individually or in combination. For example, the analysis function can be designed to filter the comparison datasets for suitable reference datasets having one or more attributes. In addition, the analysis function can be designed to ascertain or quantify a similarity or comparability of individual comparison datasets with the medical dataset. For this purpose, the analysis function can be designed to tap into and evaluate the contents of the medical dataset or of the comparison datasets respectively.

Applying the analysis function to input data can comprise in particular inputting the input data into the analysis function.

Reference datasets can be in particular those comparison datasets that, based on their contents, have a certain similarity or comparability to the medical dataset. In particular, reference datasets can be those datasets that have similar contents to the medical dataset and/or indicate similar symptoms to the medical dataset.

A document model structure can be in particular a data element that simplifies or assists producing a medical findings report for the patient to be assessed. The medical findings report can be produced in an automated manner or with the involvement of the user.

For example, the document model structure can comprise a, in particular finalized, reference findings report as such, which can serve a user as a guide. In addition, the document model structure can comprise an empty, or at least partially empty, document template, which can be populated by the user, for instance, in order to produce the findings report. In addition, the document model structure can comprise a template component, which can be combined with further components and elements in order to put together a medical findings report.

The computing facility can be a back-end computing facility. In particular, the computing facility can be a server system. The computing facility can have a cluster or group of computing facilities and data storage media. The computing facility can itself have no user interface for the user. The computing facility can be in data communication with a front-end computing facility via a medical information network, which front-end computing facility hosts a user interface for the user. The computing facility can be in data communication via the medical network with a plurality of different front-end computing facilities (but, in particular, of the same type). The front-end computing facility (facilities) can belong to a medical organization such as a medical practice, a hospital or a group of hospitals. The computing facility can likewise belong to the medical organization or be formed outside the medical organization. The computing facility can be in data communication via the medical information network with a plurality of different front-end computing facilities, which each belong to different medical organizations.

According to embodiments, the medical information network can be based on the HL7 standard. Health Level 7 (HL7) is a group of international standards for exchanging data between organizations in the healthcare sector and their computer systems. In particular, communication connections and/or data connections can be based on the FHIR standard. Fast Healthcare Interoperability Resources (FHIR) is a standard devised by HL7. It supports the exchange of data between software systems in the healthcare sector. By using the HL7 or FHIR standard, data can be transferred in a structured manner without the need for reformatting.

According to one aspect, in the step of providing the identified document model structure, the document model structure is provided to a user interface for further revision by a user. According to some examples, the user can be provided additionally with the reference dataset, or portions thereof, for the purpose of further assistance. In particular, the user can be provided with the reference findings report in addition to the document model structure (unless this report is provided anyway as the document model structure).

The user interface can be provided in particular by a front-end computing facility. In particular, the front-end computing facility can be in the form of an assessment workstation or assessment station, at which a user (in particular medical personnel such as a doctor) can open and/or view and/or analyze medical datasets, and/or at which the user can open and/or view and/or modify medical reports. The front-end computing facility can have a user interface for this purpose. The front-end computing facility can be what is known as a client.

The providing of document model structures allows elements to be provided that can be used for producing a medical findings report for the patient to be assessed, either by the user or by downstream (partially) automated processing. By identifying the provided document model structures on the basis of reference datasets, those that have proved helpful in reference cases are provided instead of any arbitrary document model structures. In other words, by searching for reference cases, a targeted selection of document model structures is made for producing a medical findings report specific to the patient to be assessed. The method can thereby employ knowledge available in an ensemble of comparison datasets for new assessment workflows, namely by providing document model structures, for instance in the form of assessment templates. In addition, this also provides the user with further diagnostic action recommendations. Overall, the user is hence given effective assistance in producing a medical findings report in an expedient and efficient manner.

According to one aspect, the step of providing the document model structure further comprises a step of anonymization of the identified document model structure.

This can guarantee sufficient protection of personal data on the patient in the particular reference dataset. This can also allow document model structures to be provided across organizational boundaries. Alternatively, the provided comparison datasets may already be anonymized.

According to one aspect, the method can further comprise a step of ascertaining an assessment context on the basis of the medical dataset and/or a user input and/or an assessment task, wherein the analysis function is further designed to ascertain the reference dataset additionally on the basis of the assessment context, and in the step of ascertaining, the at least one reference dataset is additionally ascertained by applying the analysis function to the assessment context.

The assessment context (another expression is context information) in particular can state general factors relevant to a specific diagnostic activity by the user or to the findings report to be produced. The assessment context can relate to the findings report to be produced and, for instance, stipulate what information the report is meant to contain, and, if applicable, in what order. The assessment context can comprise details about the diagnostic activity, the assessment task, the health status of the patient, etc. The assessment context can be selected for a specific diagnostic activity or assessment task from a multiplicity of elements. Examples of assessment contexts are: “Assessment of a breast CT acquisition after trauma”, “Follow-up examination as part of cancer therapy of the organ X”, “Analysis of an MR acquisition of the lungs”, “Confirmation of a suspected diagnosis Y”, etc. For example, the assessment task can be obtained on the basis of a relevant user input and/or the medical data.

Reference datasets can be selected in a more targeted manner by taking into account the assessment context. For instance, the analysis function can be designed to identify as reference datasets those comparison datasets that have an identical, or at least similar, assessment context.

According to one aspect, each document model structure is associated with an assessment context, and the step of providing comprises outputting via the user interface the assessment context associated with the identified document model structure. As a result, the user can decide more easily the assessment workflow for which the document model structure is suitable.

According to one aspect, the method further comprises a step of receiving via a user interface a user input by the user that is directed at the medical dataset, wherein the analysis function is further designed to ascertain the reference dataset additionally on the basis of user input directed at the medical dataset, and in the step of ascertaining, the at least one reference dataset is additionally ascertained by applying the analysis function to the user input.

Reference datasets can be selected in a more targeted manner by taking into account the user input. For instance, a user input can signify an aspect within the medical dataset, which aspect is relevant to the user for the assessment and can then be taken into account accordingly.

According to one aspect, the user input comprises a statement of one or more suspected diagnoses by the user. Such a user input can comprise a selection of one or more suspected diagnoses from a predetermined set (or list) of suspected diagnoses. In particular, the ascertaining of the assessment context can be based on the stated suspected diagnoses.

The reference datasets can be selected in an expedient manner by taking into account the suspected diagnoses. For example, the analysis function can be designed to match the one or more stated suspected diagnoses with diagnoses in the comparison datasets so as to ascertain suitable reference datasets. In this case, for example, the analysis function can be designed to ascertain as reference datasets those comparison datasets having diagnoses that correspond to the stated suspected diagnoses.

According to one aspect, the user input is directed at one or more of the following inputs:
defining a region of interest in the medical dataset;
detecting a medical abnormality in the medical dataset;
producing a measured value of an abnormality exhibited in the medical dataset;
selecting an analysis tool for producing a measured value of an abnormality exhibited in the medical dataset; and/or
setting one or more reproduction parameters for presenting the medical dataset in the user interface.

According to one aspect, the medical dataset comprises at least one medical image dataset of the patient to be assessed, and the user input comprises one or more of the following inputs:
defining a region of interest in the medical image dataset;
detecting a medical abnormality exhibited in the medical image dataset;
producing a measured value of a medical abnormality exhibited in the medical image dataset;
selecting an analysis tool for producing a measured value of a medical abnormality exhibited in the medical image dataset; and/or
setting one or more image reproduction parameters for presenting the medical image dataset in the user interface.

A region of interest can be understood to mean a region or data elements within the medical dataset. The region of interest can comprise at least one, but preferably a plurality of, data elements of the medical dataset. For example, this can relate to a data category that the user is currently viewing, for instance laboratory data or histopathology data. In addition, the region of interest can be a time segment of a longitudinal data series, for instance a segment of a time series of vital signs of the patient to be assessed. In addition, the region of interest can relate to a group of image elements such as pixels or voxels within medical image data contained in the medical dataset. While such a region of interest can have any shape, the region of interest is preferably circular or square. In addition, the region of interest can comprise a plurality of individual sub-regions.

A medical abnormality can relate to a data element in the medical dataset. For example, a medical abnormality can relate to corresponding image data in medical images. A medical abnormality can point to a certain condition or pathology of the patient to be assessed. The condition or pathology can be relevant to the diagnosis of the patient to be assessed.

A medical abnormality can relate to a structure or a datum that distinguishes the patient from other (healthy) patients. Medical abnormalities can be located in various organs of the patient (for instance in the lung of a patient or in the liver of a patient) or between the organs of the patient.

In particular, a medical abnormality can relate to structures or patterns exhibited in medical image data in the medical dataset. In particular, a medical abnormality can relate to a neoformation (also referred to as a “tumor”), in particular a benign neoformation, an in-situ neoformation, a malignant neoformation and/or a neoformation of uncertain/unknown behavior. In particular, a medical abnormality can relate to a nodule, in particular a pulmonary nodule. In particular, a medical finding can relate to a lesion, in particular a pulmonary lesion.

In particular, a medical abnormality can relate to an abnormal data value or a combination of abnormal data values in the medical dataset. For example, a medical abnormality can relate to a laboratory value such as a PSA value or vital signs, for instance, that deviates from a norm.

A measured value can be designed generally to quantify a medical abnormality.

In general, various types of analysis tools are available to the user. For example, such tools may be geometry measuring tools, volume measuring tools, image processing tools, outlier capture tools and/or computer-aided recognition tools. Thus the type of the analysis tool opened by the user also indicates the type of a corresponding medical abnormality that was identified using the tool. An analysis tool can be designed in particular to generate a measured value for a medical abnormality. Using this information can hence further improve the result for locating reference datasets.

According to one aspect, the reproduction parameters comprise one or more of the following parameters:
a selection of a data element from the medical dataset;
an organ segmentation applied to image data in the medical dataset;
an intensity window applied to image data in the medical dataset;
a contrast and/or brightness adjustment applied to image data in the medical dataset;
a look-up table applied to image data in the medical dataset;
an automatic view setting applied to image data in the medical dataset;
a viewing plane or direction selected for image data in the medical dataset; and/or a zoom level or pan selected for image data in the medical dataset.

Such user inputs give an indication of the regions of the medical dataset that are of interest to the user. Thus taking these into account can contribute to a targeted selection of reference datasets, and hence to providing in a targeted manner a document model structure for the further assessment. Hence, for instance, on the basis of a medical abnormality and/or an associated measured value, a systematic search can be made for reference datasets that also contain such abnormalities and/or measured values. Likewise, reproduction parameters can indicate those elements of the medical dataset that the user is currently viewing and consequently are of interest. Therefore a search can be made for reference datasets that also contain these elements.

According to one aspect, the method further comprises the steps:
providing a detection function, which differs from the analysis function, which detection function is designed to detect medical abnormalities in a medical dataset (PDS) in an automated manner; and
applying the detection function to the medical dataset by the computing facility (or system) in order to detect at least one medical abnormality, wherein
the analysis function is further designed to ascertain the reference dataset additionally on the basis of a medical abnormality detected in the medical dataset by the detection function,
in the step of ascertaining, the at least one reference dataset is ascertained additionally by applying the analysis function to the medical abnormality.

A detection function can also be referred to as a computer-aided recognition algorithm. For example, the detection function can have two stages: a recognition stage for recognizing potentially relevant patterns in medical datasets, and a classification stage for classifying the potentially relevant patterns either as potentially medically relevant or as false positives and hence results to be discarded. In general, a wealth of functions and methods are known for such computer-aided recognition and classification of candidate medical findings, all of which can be implemented in the detection function. Reference is made by way of example to the disclosure in documents US 2009/0 092 300 A1, US 2009/0 067 693 A1 and US 2016/0 321 427 A1.

According to one aspect, the medical dataset comprises at least one medical image dataset of the patient to be assessed, and the detection function is designed to detect a medical abnormality in the medical image dataset.

Employing a detection function can further assist the user. Instead of personally having to search for abnormalities, the user is provided with them in an automated manner. The analysis function can use the abnormalities in the opposite manner to search systematically for reference datasets in order to improve the hit accuracy of the provided document model structure.

According to one aspect, the method further comprises the steps:
obtaining a region of interest in the medical dataset in the computing facility;
determining an anatomical location of the region of interest by the computing facility, wherein
the analysis function is further designed to ascertain the reference dataset additionally on the basis of an anatomical location of a region of interest, and
in the step of ascertaining, the at least one reference dataset is ascertained additionally by applying the analysis function to the anatomical location.

According to examples, the region of interest can be provided by the user by way of a user input. Alternatively, the region of interest can be obtained in an automated manner by the computing facility, for instance by applying a detection function to the medical dataset.

The anatomical location (or site) can denote, for example, an organ or a portion of an organ. If, for example, a variation over time of a PSA value is identified as the region of interest, the prostate can be determined as the anatomical location.

The anatomical location can be used to retrieve information stating the anatomical context of the region of interest. This can be used to refine the group of potentially relevant reference datasets, leading to better results in providing the document model structure.

According to one aspect, the medical dataset comprises at least one medical image dataset of the patient to be assessed, and the method further comprises a step of obtaining at least one measured value of a lesion, in particular of a tumor, exhibited in the medical image dataset, wherein the analysis function is further designed to ascertain the reference dataset additionally on the basis of a measured value of a lesion, in particular of a tumor, and in the step of ascertaining, the at least one reference dataset is ascertained additionally by applying the analysis function to the measured value.

It is thereby possible to search systematically for reference datasets that exhibit lesions having similar measured values, allowing systematic identification of the document model structures.

According to one aspect, the method further comprises a step of obtaining user information concerning an attribute of the user, wherein the analysis function is further designed to ascertain the reference dataset additionally on the basis of user information, and in the step of ascertaining, the at least one reference dataset is additionally ascertained by applying the analysis function to the user information.

For example, user information can be even an identifier of the user that can be used to identify the user (for instance in the medical information network). It is thereby possible, for example, to search for reference datasets that the user has revised in the past, hence allowing systematic identification of document model structures for the user.

In addition, the user information can comprise a preference of the user with respect to one or more document model structures. It is thereby possible to ascertain systematically those reference datasets that comprise, or are based on, document model structures that correspond to the preference of the user.

In addition, the user information can comprise a statement of a user group to which the user belongs. In particular, the statement of the user group can comprise one or more guidelines on using document model structures.

For example, by taking into account the user group, the assessment can take into account guidelines that the user must follow. For instance, it can be stipulated for a user group that a medical findings report should be based on one or more document model structures. Reference datasets can accordingly be ascertained that correspond to the guidelines, guaranteeing good usability of the document model structures identified on this basis.

According to one aspect, the medical dataset comprises at least one medical image dataset of the patient to be assessed, and the method further comprises the step of determining by the computing facility at least one acquisition parameter relating to the acquisition of the medical image dataset, wherein the analysis function is further designed to ascertain the reference dataset additionally on the basis of an acquisition parameter, and in the step of ascertaining, the at least one reference dataset is ascertained additionally by applying the analysis function to the acquisition parameter.

According to one aspect, the acquisition parameter comprises one or more of the following parameters:
a patient position during the image acquisition process;
an image capture protocol used to capture raw data on the basis of which the medical image dataset was generated; and/or
an image reconstruction protocol used to generate the medical image dataset on the basis of raw data.

The image capture protocol can refer to the type of medical imaging modality used to capture the medical image dataset or the underlying raw data. For example, the capture protocol can specify whether an MRI system or a CT system was used. In addition, the capture protocol can refer to settings used for the medical imaging modality during the capture. Taking the example of an MRI system, this can be the MR pulse sequence used, for instance.

The reconstruction protocol can refer to the reconstruction algorithm and the corresponding settings used to process the acquired raw data in order to provide the medical image dataset. Taking the CT imaging process as an example, the reconstruction protocol can specify the kernel (or convolution algorithm) used. The kernel refers to the process used to modify the frequency content of projection data prior to backprojection during image reconstruction in a CT scanner. This process corrects the image by reducing blurring. The kernel affects the appearance of image structures by sharpening the image. Different kernels have been developed for specific anatomical uses, including soft tissue (standard kernel) and bones (bone kernel).

It is advantageous for the ascertaining of reference datasets to take into account acquisition parameters because the acquisition parameters provide additional insights into the organs and anatomies exhibited in the medical image dataset. Appropriate reference datasets can thus be found, allowing targeted identification of document model structures for the assessment workflow.

According to one aspect, the method further has the steps:
obtaining by the computing facility a statement of a medical abnormality in the medical dataset; and
determining by the computing facility a development over time of the medical abnormality on the basis of the medical dataset and/or further medical information on the patient to be assessed, which medical information differs from the medical dataset.

The analysis function is accordingly further designed to ascertain the reference dataset additionally on the basis of a development over time of a medical abnormality, wherein in the step of ascertaining, the at least one reference dataset is additionally ascertained by applying the analysis function to the development over time.

The medical abnormality can be obtained, for example, on the basis of a user input or by applying a detection function. The statement of the medical abnormality can comprise, for example, a manifestation of the medical abnormality in the medical dataset at a first time point.

The determining of a development over time can comprise, for example, determining at least further manifestations of the obtained abnormality at a further time point, which differs from the first time point, and determining the development over time on the basis of the at least one further manifestation, in particular on the basis of a comparison of the manifestations of the medical abnormality. In particular, measured values can be obtained from the manifestations and compared for this purpose.

By taking into account a development over time, reference datasets can be ascertained that exhibit a similar development over time of a medical abnormality. Hence document model structures can also be found that are suitable for an assessment of the development over time.

According to one aspect, the reference findings reports associated with the comparison datasets are each based on at least one, in particular standardized, template component for producing a medical findings report. In addition, the step of identifying the document model structure comprises identifying at least one template component by the computing facility on the basis of the at least one reference medical findings report associated with the at least one reference dataset. In addition, the step of providing the document model structure comprises providing the at least one identified template component.

A template component can be a component or module or model, on the basis of which a structured medical findings report can be generated (can be based). A medical findings report can be generated on the basis of one or more template components. In particular, a plurality of different template components can be combined to form a medical report.

Each template component can be specific to a particular assessment context. For example, a particular template component can be assigned to lesions in the lungs, whereas another template component is assigned to lesions in the liver.

Each template component can specify one or more data fields that must be addressed or filled in order to complete the report. In addition, a template component can comprise one or more pull-down menus containing elements that a user can select. Hence a template component can also be understood to be an input form or input mask, which structures the information to be provided for a given type of findings.

In general, the template component in the reference findings report will be populated with data specific to the case underlying the reference findings report. According to some examples, in the step of providing, the empty template component can be provided as the document model structure. For this purpose, entries in the template component can be removed automatically. Alternatively, an empty template component corresponding to the identified template component can be downloaded from a data storage medium (which can be part of the computing facility or can be in data communication therewith).

The user is thereby automatically provided with suitable template components. In return, the user is relieved of having to search in person for the correct templates in potentially large databases. The user automatically obtains a usable result that can be used directly in generating a structured finding.

According to one aspect, the method further comprises receiving via the user interface a user input by the user that is directed at editing of the identified document model structure, producing by the computing facility a medical findings report on the basis of the identified document model structure and the user input directed at the editing, and providing by the computing facility the medical findings report. A complete medical findings report can thereby be created through continuous human-machine interaction.

According to one aspect, the method further comprises editing and/or pre-populating the identified document model structure on the basis of the medical dataset and/or the at least one reference dataset (and/or on the basis of a user input directed at the medical dataset, and/or a medical abnormality captured by the detection algorithm), wherein in the step of providing, the pre-populated document model structure is provided to the user for further revision.

According to one aspect, the method further comprises editing and/or populating the identified document model structure on the basis of the medical dataset and/or the at least one reference dataset (and/or on the basis of a user input directed at the medical dataset, and/or a medical abnormality captured by the detection algorithm) in order to generate a final medical findings report in an automated manner.

Medical findings reports can thereby be provided automatically without a user being involved. This can relieve the load on users, who can hence have more time, for example, to devote to complex cases in which no reference datasets can be found.

According to one aspect, the method further comprises a step to check whether at least one reference dataset can be found with sufficient confidence (or similarity measure). If so, a final medical findings report can be generated in an automated manner on the basis of the document model structure. If not, the document model structure can be provided to the user for further revision.

According to one aspect, the at least one identified document model structure in the reference findings report is populated with an assessment text containing natural language, and the method further comprises the steps:
providing a language analysis algorithm, which is designed to adapt an assessment text to a medical dataset by evaluating the assessment text and the medical dataset;
adapting the assessment text to the medical dataset by applying the language analysis algorithm to the assessment text and the medical dataset by the computing facility;
pre-populating the identified document model structure with the adapted assessment text by the computing facility, wherein
in the step of providing, the pre-populated document model structure is provided.

For example, the language analysis algorithm can be designed to recognize and assign a meaning to individual elements in the assessment text. Various known methods and functions can be used to do this. For example, this can be done via latent semantic indexing (LSI for short).

In other words, an automated evaluation of unstructured contents of prior findings can be achieved by employing a language analysis algorithm. This can furnish the user with further information relevant to the assessment.

According to one aspect, the analysis function is designed to calculate a similarity measure between a medical dataset and a comparison dataset, which similarity measure states a similarity between the medical dataset and the comparison dataset. In addition, the step of ascertaining the at least one reference dataset comprises calculating a similarity measure for each of the comparison datasets by applying the analysis function to the medical dataset and the comparison datasets, and the reference datasets are ascertained from the comparison datasets on the basis of the determined similarity measures.

A similarity measure can be determined for each of the comparison datasets being considered, which similarity measure is based on a similarity between the medical dataset and the particular comparison dataset, and in particular states or quantifies a similarity. For example, a similarity measure can be a numerical value or “score”. The similarity measures can be determined, for example, on the basis of applying a similarity metric, which outputs on the basis of the input quantities, i.e. the medical dataset and a comparison dataset, a similarity measure. In particular, said similarity metric can be implemented in the analysis function. Reference datasets are in particular those comparison datasets that have a certain similarity to the medical dataset. In other words, reference datasets can be in particular those comparison datasets that have a similarity measure lying above a predetermined or defined or definable threshold.

The calculation of similarity measures provides an objective and reproducible criterion that can be used to locate reference datasets.

According to one aspect, the analysis function is designed to extract a data descriptor from a medical dataset, and/or a corresponding data descriptor from a comparison dataset, which data descriptors state attributes of the underlying medical dataset and/or of a comparison dataset that are relevant to ascertaining reference datasets. The analysis function is further designed to calculate a similarity measure between a medical dataset and a comparison dataset on the basis of their data descriptors, in particular by inputting their data descriptors into a similarity metric. In addition, the calculating of a similarity measure for a comparison dataset comprises the steps:
obtaining a data descriptor from the medical dataset in particular by applying the analysis function to the medical dataset;
obtaining a corresponding data descriptor for the one comparison dataset in particular by applying the analysis function to the one comparison dataset; and
calculating the similarity measure for a comparison dataset on the basis of the data descriptor and the particular corresponding data descriptor by applying the analysis function to the data descriptor and the particular corresponding data descriptor.

A data descriptor can have one or more features that have been extracted or calculated from the medical dataset, and in particular from the image data of the medical dataset. In addition, the data descriptor can be based on (or take additional account of) further information or non-image data, for instance metadata on the image data, patient data, further measured values, medical findings reports, etc. The expression “feature signature” can be another term for data descriptor. In particular, the data descriptor can characterize the medical dataset. The features of the data descriptor can be combined into a feature vector. In particular, the data descriptor can have such a feature vector. Features extracted from image data can be morphological and/or structural features and/or features relating to a texture and/or to a pattern. Features extracted from non-image data can be features relating to a diagnosis, to a measured value, to demographic information, etc. The analysis function can be designed in particular to ascertain similarity measures on the basis of the data descriptor. The analysis function can have different modules for extracting data descriptors, for instance image analysis modules for processing medical image data or language analysis modules for processing medical findings reports.

The ascertaining of the similarity measures can comprise extracting or receiving a corresponding data descriptor from each of the possible comparison datasets. This can be done in the same way as for the data descriptor extracted on the basis of the medical dataset. The ascertaining of the similarity measure can further comprise comparing each of the corresponding data descriptors with the data descriptor.

The step of comparing can be based in particular on the evaluation of a similarity metric for two data descriptors. A similarity metric can be designed to quantify a distance between the respective data descriptors in the coordinate system of the data descriptors. Similarity metrics can be defined. For example, a similarity metric can be based on calculating a cosine similarity of the data descriptors and/or calculating a weighted sum of the difference or similarity of individual features of the data descriptors. In particular, those comparison datasets that have an associated similarity measure greater than a defined or definable threshold can be identified as reference datasets.

By using data descriptors, easy-to-implement and easily transferable parameters for matching of different datasets are defined. In addition, the features contained in the feature signatures can be based on higher-level observables derived from the datasets, which observables often characterize the attributes of the datasets better than the underlying data itself.

According to one aspect, the analysis function comprises a trained function.

A trained function generally maps input data onto output data. Said output data may depend in particular on one or more parameters of the trained function. Other terms for trained function are trained mapping rule, mapping rule containing trained parameters, function containing trained parameters, algorithm based on artificial intelligence, and machine-learning algorithm.

An example of a trained function is an artificial neural network. The term “neural net” can also be used instead of the term “neural network”. A neural network is basically built like a biological neural net—for instance a human brain. In particular, an artificial neural network comprises an input layer and an output layer. It can further comprise a plurality of layers between input layer and output layer. Each layer comprises at least one node, preferably a plurality of nodes. Each node can be understood as a biological processing unit, for instance as a neuron. In other words, each neuron is equivalent to an operation applied to input data. Nodes in one layer can be connected to nodes in other layers by edges or connections, in particular by directional edges or connections. These edges or connections define the data flow between the nodes of the network. The edges or connections are associated with a parameter, often referred to as a “weight” or “edge weight”. This parameter can regulate the importance of the output from a first node for input to a second node, with the first node and the second node being connected by an edge. In particular, a trained function can also be a deep artificial neural network (or a deep neural network).

According to one aspect, the trained function has a convolutional neural network and in particular a region-based convolutional neural network.

In particular, the convolutional neural network can be a deep convolutional neural network. In this case, the neural network has one or more convolutional layers and one or more deconvolutional layers. In particular, the neural network can comprise a pooling layer. By using convolutional layers and/or deconvolutional layers, a neural network can be employed particularly efficiently for image processing because, despite numerous connections between node layers, only a few edge weights must be determined (namely the edge weights corresponding to the values of the convolution kernel). This can also improve the accuracy of the neural network for the same amount of training data.

A trained function learns by adapting parameters that determine the mapping of input data onto output data. In the case of neural networks, these are, for example, the weights or weighting parameters (e.g. of the edge weights) of individual layers and nodes. A trained function can be trained by supervised learning methods, for example. The method of backpropagation can be used here, for instance. During the training, the trained function is applied to training input data in order to generate corresponding output values, the target values of which are known in the form of training output data. The difference between the output values and the training output data can be used to implement a cost functional or loss functional as a measure of how well or how poorly the trained function is fulfilling the task it has been set. The aim of the training is to find a (local) minimum of the cost functional by iteratively adapting the parameters (e.g. the edge weights) of the trained function. This ultimately makes the trained function capable of delivering acceptable results over a (sufficiently) large cohort of training input data. This optimization problem can be performed using a method known as stochastic gradient descent or other approaches known in the specialist field.

According to embodiments, training datasets would each have medical training datasets and training comparison datasets, and, depending on the configuration of the trained function, associated verified reference datasets and/or verified (in particular relative) similarity measures. The verified reference datasets could be based here on an annotation that a user had made on the basis of an analysis or assessment of the medical training dataset. The same applies to the verified similarity measures.

Training of the trained function could hence then comprise, according to some embodiments, applying the trained function to the medical training datasets and/or training comparison datasets in order to generate output values, and comparing the output values with verified reference datasets and/or verified similarity measures. One or more parameters of the trained function can then be adapted on the basis of the comparison.

According to one aspect, the trained function has:
an encoder segment, which is designed to extract a data descriptor from a medical dataset, and/or a corresponding data descriptor from a comparison dataset;
a classifier segment, which is designed to calculate a similarity measure between a medical dataset and a comparison dataset on the basis of a data descriptor of the medical dataset and a corresponding data descriptor of the comparison dataset.

According to one aspect, the encoder segment is a, in particular convolution, neural network. According to one aspect, the classifier segment comprises a support vector machine, a k-nearest neighbors algorithm, a decision tree algorithm, and/or a Naive Bayes algorithm.

According to one aspect, the trained function is further designed to calculate a similarity measure by applying a machine-learned similarity metric to data descriptors, wherein the calculating of the similarity measure comprises applying the machine-learned similarity metric to the data descriptor and the particular corresponding data descriptor.

According to one aspect, the machine-learned similarity metric is implemented in the classifier segment.

According to one aspect, in the step of identifying at least one document model structure, a plurality of document model structures are identified for selection by the user, wherein in the step of providing, the plurality of identified document model structures are provided to the user via the user interface. In addition, the method comprises receiving via the user interface a user input by the user that is directed at selecting at least one document model structure, wherein in the step of providing, the at least one document model structure selected by the user input directed at the selection of the at least one document model structure is provided for further revision by the user.

By the user selection, suitable document model structures can be provided through human-machine interaction. The user can thereby flexibly influence the processing in order to improve the result.

According to one aspect, the method comprises receiving via the user interface a user input by the user that is directed at selecting the identified at least one document model structure for further revision. In particular, the user input can be directed at confirming or discarding the identified at least one document model structure.

According to one aspect, the method further comprises adapting the analysis function on the basis of the user input directed at the selection of at least one document model structure.

In other words, the analysis function can thereby be improved further on the basis of the user interaction. In particular, a trained function contained in the analysis function can be trained further in order to facilitate improved ascertainment of reference datasets and hence targeted provision of document model structures.

According to one aspect, a system is provided for providing a document model structure for producing a medical findings report in the assessment of a patient to be assessed. The system has a computing facility and an interface. The interface is designed to receive a medical dataset of the patient to be assessed and a plurality of comparison datasets, which differ from the medical dataset, wherein each comparison dataset has at least one reference medical findings report. The computing facility is designed to host an analysis function which is designed to ascertain for a medical dataset at least one reference dataset from a plurality of comparison datasets, to ascertain at least one reference dataset from the plurality of comparison datasets by applying the analysis function to the medical dataset and the comparison datasets, to identify, on the basis of the at least one reference medical findings report associated with the at least one reference dataset, at least one document model structure for the patient to be assessed, and to provide the identified at least one document model structure via the interface.

The interface can be designed generally for data exchange between the computing facility and further components. The interface can be implemented in the form of one or more individual data interfaces, which can have a hardware and/or software interface, for example a PCI bus, a USB interface, a FireWire interface, a ZigBee interface or a Bluetooth interface. The interface can also have an interface to a communication network, which communication network can have a local area network (LAN), for example an intranet or a wide area network (WAN), or can have an internet. The one or more data interfaces can accordingly have a LAN interface or a wireless LAN interface (WLAN or Wi-Fi).

According to one aspect, a system for providing a medical report is also provided, which system has the aforementioned apparatus and at least one user interface which is designed to receive via the interface and to display to a user the provided document model structures.

In addition, the system can have a data storage medium, which has a connection to the interface and in which the comparison datasets and/or the analysis function and/or the medical dataset are stored and can be retrieved and hence provided via the interface. Said data storage medium can be in the form of a central or local storage unit or Cloud storage.

In addition, the system can comprise one or more imaging modalities, for instance a computed tomography system, a magnetic resonance system, an angiography system, an X-ray system, a positron emission tomography system, a mammography system, and/or a system for generating histopathology image data.

The advantages of the proposed systems are essentially the same as the advantages of the proposed methods. Features, advantages or alternative embodiments/aspects of the methods can be applied likewise to the other claimed subject matter, and vice versa.

In a further aspect, embodiments of the present invention relate to a computer program product, which comprises a program and can be loaded directly into a memory of a programmable controller, and has program instructions and/or means, e.g. libraries and auxiliary functions, in order to perform a method for providing a medical report, in particular according to the aforementioned embodiments/aspects, when the computer program product is executed.

In a further aspect, embodiments of the present invention also relates to a computer-readable storage medium, on which are stored readable and executable program segments in order to perform all the steps of a method for providing a medical report according to the aforementioned embodiments/aspects when the program segments are executed by the controller.

Said computer program products can comprise software containing a source code, which still needs to be compiled and linked or just needs to be interpreted, or an executable software code, which for execution only needs to be loaded into the processing unit. The methods can be performed quickly, identically reproducibly and robustly by the computer program products. The computer program products are configured such they can perform the method steps according to embodiments of the present invention via the computing unit. Therefore, the computing unit must have the necessary specification in each case, such as, for example, a suitable main memory, a suitable processor, a suitable graphics card or a suitable logic unit, in order to be able to perform the respective method steps efficiently.

The computer program products are stored, for example, on a computer-readable storage medium or on a network or server, from where they can be loaded into the processor of the particular computing unit, which processor may be connected directly to the computing unit or may form part of the computing unit. In addition, control information of the computer program products can be stored on a computer-readable storage medium. The control information on the computer-readable storage medium can be configured such that it performs a method according to embodiments of the present invention when the data storage medium is used in a computing unit. Examples of computer-readable storage media are a DVD, a magnetic tape or a USB stick, on which is stored electronically readable control data, in particular software. When this control data is read from the data storage medium and stored in a computing unit, all the embodiments/aspects according to the present invention of the above-described methods can be performed. Hence embodiments of the present invention can also proceed from said computer-readable medium and/or from said computer-readable storage medium. The advantages of the proposed computer program products or of the associated computer-readable media are essentially the same as the advantages of the proposed methods.

### BRIEF DESCRIPTION OF THE DRAWINGS

Further features and advantages of the present invention are disclosed in the following explanations of exemplary embodiments with reference to schematic drawings. Modifications mentioned in this connection can be combined with one another to form new embodiments. The same reference signs are used for the same features in different figures, in which:

FIG. 1 is a schematic representation of an embodiment of a system for providing a document model structure for producing a medical findings report;

FIG. 2 is a flow diagram of a method for providing a document model structure for producing a medical findings report according to one embodiment;

FIG. 3 is a data flow diagram of a method for providing a document model structure for producing a medical findings report according to one embodiment;

FIG. 4 is a diagram of optional method steps in a method for providing a document model structure for producing a medical findings report according to one embodiment;

FIG. 5 is a diagram of optional method steps in a method for providing a document model structure for producing a medical findings report according to one embodiment;

FIG. 6 is a diagram of optional method steps in a method for providing a document model structure for producing a medical findings report according to one embodiment;

FIG. 7 is a flow diagram of a method for providing a trained function for identifying a reference dataset; and

FIG. 8 is a schematic representation of an embodiment of a system for providing the trained function.

### DETAILED DESCRIPTION

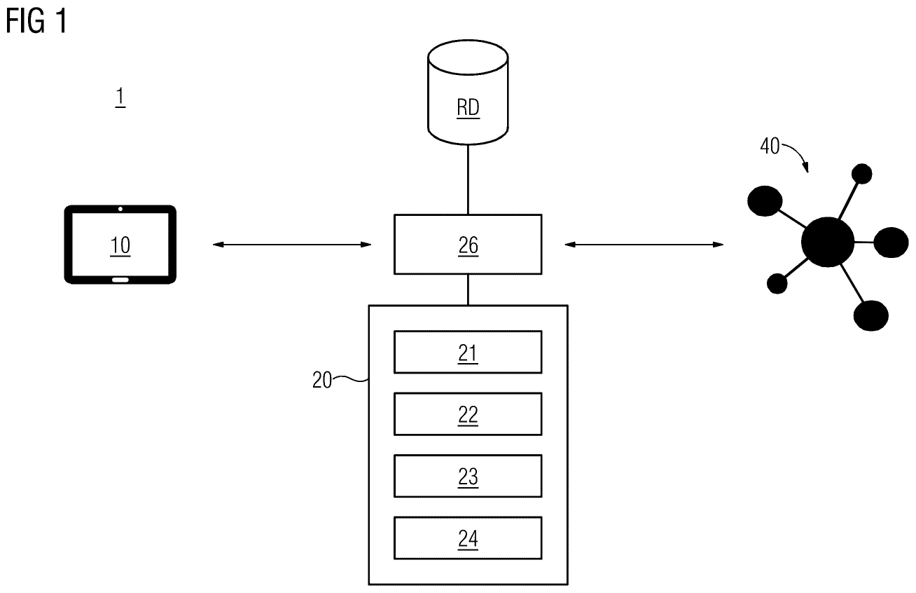

FIG. 1 shows a system 1 for providing a document model structure DVS or for providing a medical findings report MBB according to one embodiment. The system 1 has at least one front-end computing facility 10 having a user interface (or also just “user interface 10”), a storage facility RD (or “storage device RD” or “storage apparatus RD”) and a back-end computing facility 20 (or also just “computing facility 20,” “computing system 20” or “computing apparatus 20”), which are in communication with one another via a medical network or a data interface 26.

The medical dataset PDS and further information can be provided to the front-end computing facility 10 via suitable interfaces 26 by the storage facility RD or by the medical information system 40. Typically, a system as shown in FIG. 1 has a plurality of front-end computing facilities 10, which all access the same medical information system 40 and are in data communication with the back-end computing facility 20. In the embodiment shown, the storage facility RD, the back-end computing facility 20, the medical information system 40 and the front-end computing facility (facilities) 10 are part of the same medical organization. A medical organization can be a medical practice, a practice group, a hospital, or a group of hospitals, for example. The network connecting these components via the interface 26 can accordingly be an internal network of the organization and, for example, comprise an intranet (for instance a local area network and/or a wireless local area network).

The front-end computing facility 10 can be in the form of an assessment station or assessment workstation, for example, at which a user can view and analyze patient data or a medical dataset PDS and also produce, check, modify and appraise medical findings reports MBB. The front-end computing facility 10 can have a user interface for this purpose, for instance comprising a display and/or an input facility. The front-end computing facility 10 can have a processor. The processor can have a central processing unit (CPU), a graphics processing unit (GPU), a digital signal processor (DSP), an image processing processor, an integrated circuit (digital or analog), or combinations of the aforementioned components, and further facilities for providing a medical findings report MBB according to embodiments. The front-end computing facility 10 can comprise, for example, a desktop PC, laptop or a tablet.

The medical information system 40 can be designed generally for capturing and/or storing and/or forwarding medical datasets PDS. For example, the medical information system 40 can have one or more databases (not shown). In particular, the databases can be realized in the form of one or more Cloud storage modules. Alternatively, the databases can be realized as a local or distributed storage medium, for instance as a PACS (Picture Archiving and Communication System), a hospital information system (HIS) a laboratory information system (LIS), an electronic medical record (EMR) information system, and/or further medical information systems. According to some examples, the medical information system 40 can also comprise one or more medical imaging modalities (not shown), for instance a computed tomography system, a magnetic resonance system, an angiography system, a C-arm X-ray system, a positron emission tomography system, a mammography system, an X-ray system or the like.

The storage facility RD can be in the form of a central or local database. The storage facility RD in particular can be part of a server system. The storage facility RD in particular can be part of the medical information system 40. The storage facility RD is designed to store a number of comparison datasets VDS. The storage facility RD can also be referred to as a data source or database. In addition, a plurality of predetermined document model structures DVS can be stored in the storage facility 26.

The storage facility RD can be designed to provide comparison datasets VDS and/or document model structures VDS centrally for the system 1 and in particular for different front-end computing facilities 10.

A document model structure DVS can be considered in embodiments to be a component for a medical findings report MBB. Document model structures DVS can be designed for revision by the user via the front-end computing facility 10. In addition, document model structures DVS can be designed to be contained in a medical findings report MBB. Document model structures DVS can comprise one or more data fields DF, into which patient-specific information and/or the underlying medical findings can be entered. The data fields DF can be empty fields or placeholders for various types of data such as text, measured values or images. According to some examples, document model structures DVS comprise one or more pull-down menus containing entries from which a user can select.

A document model structure DVS can be specific to a particular assessment context. In other words, document model structures DVS can be different for different assessment contexts. For example, they can differ in the number and type of the data fields DF.

The medical dataset PDS can have medical image data and/or other medical data that does not comprise image information. In this context, image data can relate to medical image data having two or three spatial dimensions. Furthermore, the image data can additionally have a time dimension. For example, the image data can have been generated using a medical imaging modality, for instance an X-ray, computed tomography, magnetic resonance, positron emission tomography or angiography device, or further devices. Such image data can also be referred to as radiology image data.

In addition, the medical dataset PDS can also comprise histopathology image data exhibiting one or more histopathology images. Histopathology image data is image data that is based on a tissue sample from a patient. Tissue sections are prepared from the tissue sample and stained with a histological stain. The tissue sections prepared in this way are then digitized to obtain the histopathology image data. Specialized scanners known as slide scanners can be used for this purpose. The image acquired in this process is also referred to as a whole slide image. The acquired image data is typically two-dimensional pixel data.

The image data contained in the medical dataset PDS can be formatted in accordance with the DICOM format, for example. DICOM (=Digital Imaging and Communications in Medicine) is an open standard for the communication and administration of medical image data and associated data.

In addition to image data, the medical dataset PDS can also comprise non-image data. Non-image data can be, for example, examination results that are not based on medical imaging. This can comprise laboratory data, vital-signs data, spirometry data or the logs from neurological examinations. In addition, non-image data can comprise text datasets, for instance structured and unstructured medical findings reports (also called medical reports). In addition, non-image data can also be patient-related data. This can comprise, for example, demographic details about the patient, for instance relating to age, gender or body weight. The non-image data can be incorporated in the image data, for instance as metadata. Alternatively or additionally, the non-image data can also be held in an electronic medical record (EMR for short) of the patient, i.e. separately from the image data. Such electronic medical records can be archived in the medical information system 40, for example.

The comparison datasets VDS constitute datasets that have already been assessed. In other words, these comparison datasets VDS each have at least one (finalized) medical findings report, the reference findings report R-MBB as it is called.

According to embodiments of the present invention, each comparison dataset VDS can also have just one reference findings report R-MBB. In other words, the comparison datasets can each be a reference findings report, or each consist of a reference findings report R-MBB.

According to other exemplary embodiments, comparison datasets VDS can also have the same structure as the medical datasets PDS, i.e. comprise in addition to the reference findings report in particular also medical image data and non-image data.

The back-end computing facility 20 can have a processor. The processor can have a central processing unit (CPU), a graphics processing unit (GPU), a digital signal processor (DSP), an image processing processor, an integrated circuit (digital or analog), or combinations of the aforementioned components, and further facilities for providing a document model structure DVS according to embodiments. The back-end computing facility 20 can be implemented as an individual component or have a group of computers, for instance a cluster. Such a system can be called a server system. Depending on the embodiment, the back-end computing facility 20 can be a local server. In addition, the back-end computing facility 20 can have a main memory such as a RAM, for instance in order to store temporarily the patient data PD, data filters DF, individual information EI, or report templates BT. The back-end computing facility 20 is designed, for instance via computer-readable instructions, by design and/or hardware, such that it can execute one or more method steps according to embodiments of the present invention.

The back-end computing facility 20 can be in communication via the interface 26 with the front-end computing facility 10 and/or the storage facility RD and/or the medical information system 40. Via this interface 26, the back-end computing facility 20 can receive medical datasets PDS, comparison datasets VDS, and/or user inputs, on the basis of which a document model structure can be automatically selected, and a medical findings report MBB computer-generated and provided.

The back-end computing facility 200 can have various modules available for providing a medical report MB.

Module 21 is a data retrieval module. It is designed to access the medical information system 40 or the storage facility RD, and to search for the medical datasets PDS or for comparison datasets VDS. In particular, the module 21 can be designed to formulate search queries and parse these queries for the medical information system 40.

Module 22 can be a user interaction module or unit. The module 22 can be designed to provide the user with a document model structure DVS for further revision. In addition, the module 22 can be designed to capture and provide to the processing in the back-end computing facility one or more user inputs. Such user inputs can comprise, for example, voice, gestures, eye movements, handling of input devices such as a computer mouse, etc. The user inputs can be directed at interaction with the medical dataset PDS or relate to the production of a medical findings report MBB on the basis of the document model structure DVS.

Module 23 is designed to provide reference datasets RDS. Module 23 can also be referred to as a similarity analysis module. For this purpose, module 23 can be designed to access the provided comparison datasets VDS and to select one or more reference datasets RDS. The reference datasets RDS are characterized in that they have a certain similarity to the medical dataset PDS or to the medical circumstances or assessment context of the medical dataset PDS. For this purpose, module 23 can be designed, for example, to extract a data descriptor from the medical dataset PDS and to compare this data descriptor with respective data descriptors from the comparison datasets VDS. A data descriptor can be regarded in this context as a data vector or feature vector, in which are aggregated relevant features for comparisons of different medical datasets. Said relevant features can be extracted both from the image data and from the non-image data of the medical dataset PDS, or, for example, comprise demographic information about the particular patient (such as age or gender), laboratory values (such as a PSA value for the patient), and/or vital-signs data from the patient. Features extracted from image data can comprise, for example, image information such as patterns, color information, intensity values, etc. The module 23 can compare the data descriptor generated from the medical dataset PDS with respective data descriptors of the comparison datasets VDS in order to ascertain amongst the comparison datasets VDS cases that have a certain similarity to the medical dataset PDS of the patient to be assessed. For this purpose, the module 23 can ascertain for each of the comparison datasets VDS considered a similarity measure that states (or quantifies) a similarity of the medical dataset PDS to the particular comparison dataset VDS. Comparison datasets VDS having a certain similarity measure (e.g. a similarity measure above a certain threshold value) are identified as reference datasets RDS.

In order to perform the similarity analysis, the module 23 can be designed to execute an analysis function AF. The analysis function AF is then designed to identify, on the basis of the medical dataset PDS and the comparison datasets VDS, one or more reference datasets RDS, and to provide respective similarity measures. The analysis function AF can have one or more trained functions.

The module 24 can be a reporting module, which is used to produce a medical findings report MBB. In particular, the module 24 can be designed to request suitable document model structures DVS from the storage facility RD. In addition, the module 24 can be designed to edit the document model structures DVS on the basis of user inputs. In addition, the module 24 can be designed to combine a plurality of document model structures DVS into a medical findings report.

The partitioning of the back-end computing facility 20 into modules 21-24 is used here merely as a way of explaining more easily the manner of operation of the back-end computing facility 20 and is not to be understood in a restrictive sense. The modules 21-24 or their functions can also be combined in one element. The modules 21-24 can be also be regarded in this context as computer program products or computer program segments which, on execution in the back-end computing facility 20, realize one or more of the method steps described herein.

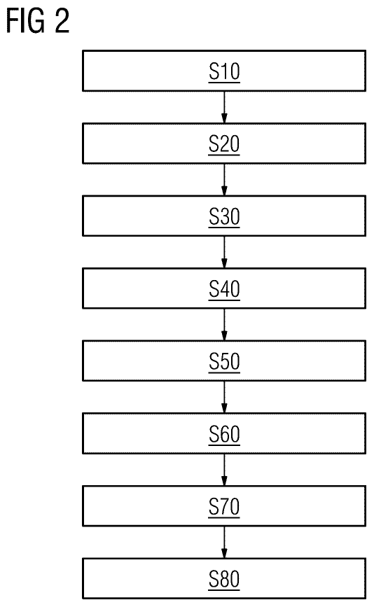

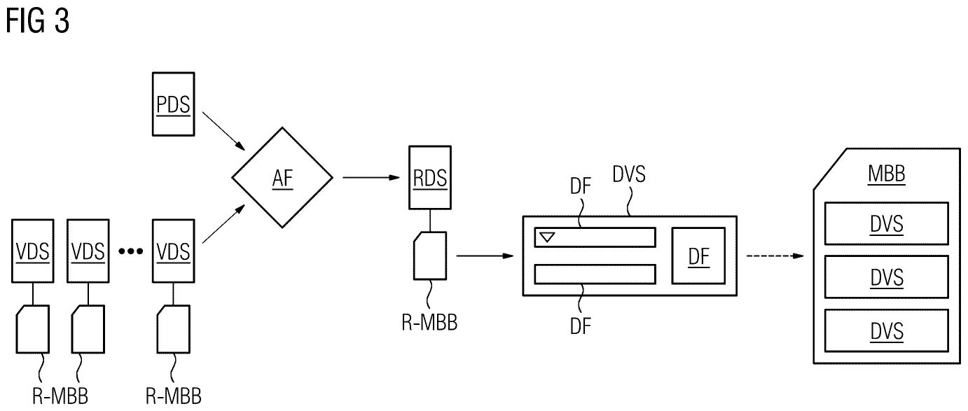

FIG. 2 depicts a schematic flow diagram of a method for providing a document model structure DVS or a medical findings report MBB. The order of the method steps is limited neither by the sequence shown nor by the chosen numbering. Thus the order of the steps can be interchanged if applicable, and individual steps can be omitted. Moreover, one or more steps, in particular a sequence of steps, and optionally the entire method, can be executed repeatedly. FIG. 3 shows an associated diagram that depicts by way of example data streams associated with the method shown in FIG. 2.

In some embodiments, the method depicted in FIG. 2 is aimed at offering, on the basis of an automated analysis of the data available on a patient, a selection of possible document model structures DVS, and, if applicable, refining said selection by continued human-machine interaction. The available data is given by the medical dataset PDS of the patient to be assessed and by comparison datasets VDS.

In a first step S10, the medical dataset PDS of the patient to be assessed is provided. This can comprise a user manually selecting the particular case via the front-end computing facility 10. In addition, this can comprise loading the medical dataset PDS from the medical information system 40. Step S10 can also comprise the back-end computing unit 20 receiving the medical dataset PDS.

According to an idea of the present invention, the back-end computing facility 20 automatically compares the medical dataset PDS with the comparison datasets VDS in order to identify amongst the comparison datasets VDS datasets that have a certain similarity to the medical dataset PDS. These similar datasets are also called reference datasets RDS. Assuming that similar medical datasets imply similar medical findings reports MBB, possible document model structures DVS for the medical dataset PDS to be assessed can be inferred from the already known reference findings reports R-MBB of the reference datasets RDS.

In step S20, comparison datasets VDS are accordingly provided. The comparison datasets VDS can be provided to the back-end computing facility 20 for example via the storage facility RD or from a storage facility within the medical information system 40.

In step S30, the back-end computing facility 10 is provided with an analysis function AF, which is designed to find from the comparison datasets VDS one or more reference datasets RDS for the medical dataset PDS. For example, the analysis function AF can be provided by being kept available as executable computer code in a memory of the back-end computing facility 20.

In step S40, the analysis function AF is used to ascertain from the comparison datasets VDS one or more reference datasets RDS, which preferably have a certain similarity to the medical dataset PDS.

Step S40 can be based on a similarity analysis in which similarities between the medical dataset PDS and the comparison datasets VDS are identified or quantified by the analysis function AF. The similarities can be expressed or quantified here by similarity measures. In particular, the analysis function AF can ascertain for each of at least a subset of the comparison datasets VDS a similarity measure, which expresses a similarity of the particular comparison dataset VDS in each case with the medical dataset PDS.

All the comparison datasets VDS having a certain degree of similarity to the medical dataset PDS can be identified as reference datasets RDS that are potentially relevant to the assessment of the medical dataset PDS. For example, all those comparison datasets VDS that have a similarity measure above a defined or definable threshold can be identified as reference datasets RDS. Alternatively, the comparison datasets VDS having the comparatively highest similarity measure can be identified as reference datasets RDS.

Thus step S40 leads to one or more reference datasets RDS, as shown in FIG. 3. Each reference dataset RDS has an associated reference findings report R-MBB.

In step S50, one or more document model structures DVS are identified on the basis of the reference findings report(s) R-MBB of the reference datasets RDS. For example, the document model structure DVS on which the reference findings reports R-MBB are based can be recognized for this. The document model structures DVS recognized in this way can then be identified as relevant to the case to be assessed.

In step S60, the document model structures DVS identified in this way are provided, either to a user at the front-end computing facility 10 or to an automated process (for example in the back-end computing facility 20) for further processing, in order to produce on the basis of the document model structure DVS a medical findings report MBB for the patient to be assessed. For example, in step S60, an “empty” version of the document model structure DVS can be downloaded from the storage facility RD and provided. Alternatively, the document model structure DVS edited and/or populated for the reference findings report R-MBB can be provided.

In an optional step S70, a medical findings report MBB is generated on the basis of the document model structure DVS. This can comprise receiving user inputs that are directed at revising the document model structure DVS via the front-end computing facility 10. This can comprise entering content into the document model structure DVS or altering content of the document model structure DVS. For the medical findings report MBB, the document model structure DVS revised in this way by the user can additionally be combined with other document model structures DVS relating to other medical findings MF. The resultant medical findings report MBB can then be forwarded to other systems or users, for instance via the medical information system 40. In addition, the resultant medical findings report MBB can be archived, for instance in the report database RD or in the medical information system 40. In particular, the medical findings report MBB produced in this way can be stored as a further comparison dataset VDS in order to expand the “knowledge” in the system 1.

The optional step S80 provides human-machine interaction for selecting a document model structure. Hence, for instance, a plurality of different document model structures can be provided in step S50, which can then be provided to the front-end computing facility 10 in step S60. In step S80, a user input can consequently be received that is directed at selecting one or more of the provided document model structures DVS and/or discarding others. The selected document model structures can then be used further to produce the medical findings report MBB.

According to some implementations, the user inputs can also be fed back to the analysis function AF in order to improve this function, for instance by further training of same.

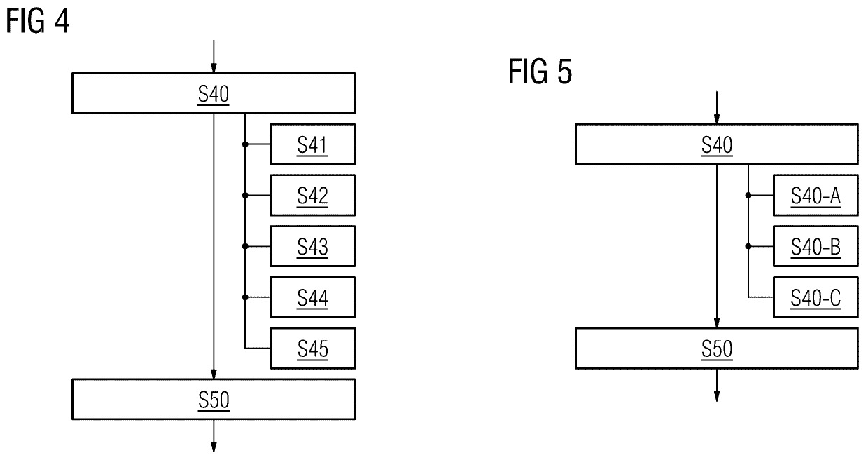

FIG. 4 depicts further optional steps by which additional information about the case to be assessed or the assessment workflow can be obtained and used for determining the comparison datasets VDS or identifying the document model structures DVS. The steps can be incorporated individually or in any combination into the method shown in FIG. 2 or further methods described herein. In particular, the steps shown in FIG. 4 can be executed individually or in any combination as part of step S40.

The optional step S41 is directed at including a user input. The user input can be input into the front-end computing facility 10 by a user and received in the back-end computing facility 20. The user input is preferably an input by the user that this user directs at the medical dataset PDS as part of an analysis or assessment.

For example, the user input can be directed at a selection of a certain portion of the medical dataset PDS, for instance image data or laboratory data inside the medical dataset PDS. In addition, the user input can comprise marking a relevant report or region of interest within a portion of the medical dataset PDS. For instance, the user input can be directed at defining a segment from medical image data or defining individual values in a time series of values of the patient. In addition, the user input can relate to one or more reproduction parameters used to present the medical dataset, or portions thereof, in the front-end computing facility 10. Furthermore, the user input can be directed at producing a measured value on the basis of the medical dataset PDS. For example, the user can do this by selecting a digital measurement tool in the front-end computing facility 10 and applying said tool to the medical dataset PDS. For example, the user can activate a tool for measuring medical image data in order to measure the geometry of anatomic features shown in the image data.

The aforementioned and further user inputs give an indication of the information in the medical dataset PDS that the user deems relevant to the assessment. The analysis function AF can accordingly be designed to take into account the user inputs in ascertaining the comparison datasets VDS. For example, such user inputs can be used by the analysis function AF to filter out automatically the corresponding constituent parts of the medical dataset PDS. These constituent parts can then be used, or at least weighted more heavily, in the identification of comparison datasets VDS. If, for example, via a user input the user has identified as relevant certain vital-signs data of the patient to be assessed, the analysis function AF can be designed to search systematically for those comparison datasets VDS that have comparable vital-signs data.

Said relevant regions can also be detected automatically according to further exemplary embodiments. In step S42 a detection function can be provided for this purpose, for example, which is designed to recognize medical abnormalities automatically in a medical dataset. For example, the detection function can be designed to capture in an automated manner suspect vital signs and/or conspicuous patterns in medical image data. Such detection functions are known in principle and can comprise what are known as outlier capture algorithms or image analysis functions, which can be based in particular on trained functions. For example, the detection function can be provided by being kept available as executable computer code in a memory of the back-end computing facility 20.

In step S43, the detection function is applied to the medical dataset PDS, whereby at least one medical abnormality is detected in the medical dataset PDS.

Similar to that in step S41, the analysis function AF is designed to take the automatically captured medical abnormality into account in ascertaining the reference datasets RDS and thereby improve the “hit accuracy” of the reference datasets. The medical abnormality is accordingly input (together with the medical dataset PDS) into the analysis function AF.

A development over time of a medical abnormality can give a further indication of appropriate reference datasets. For example, reference datasets RDS having similar developments over time can indicate similar pathologies and hence appropriate reference findings reports R-MBB.

First, in step S44, a medical abnormality is obtained, either by using a detection function as in steps S42 and S43, or on the basis of a user input as in step S41.

Then in step S45, a development over time of the obtained medical abnormality is calculated. This can be done, for example, by searching in an automated manner for earlier manifestations of the medical abnormality in the medical dataset PDS or in further available data of the patient to be assessed. For example, if a lesion in a medical image dataset was obtained as the medical abnormality, further medical image data of the patient can be opened and analyzed for the lesions. In addition, a measured value characterizing the lesion can be generated for the different time points, whereby a development over time of the medical abnormality can be provided.

Similar to that in step S41, the analysis function AF is designed to take the development over time of the medical abnormality into account in ascertaining the reference datasets RDS and thereby improve the “hit accuracy” of the reference datasets RDS. The medical abnormality is accordingly input (together with the medical dataset PDS) into the analysis function AF.

FIG. 5 shows an exemplary embodiment of individual steps for ascertaining reference datasets RDS. In particular, the individual steps can be executed within step S40. The order of the method steps is limited neither by the sequence shown nor by the chosen numbering. Thus the order of the steps can be interchanged if applicable, and individual steps can be omitted. Moreover, one or more steps, in particular a sequence of steps up to and including all the steps, can be executed repeatedly.

The steps shown in FIG. 5 are aimed at ascertaining for each comparison dataset VDS a similarity measure, which quantifies, for instance in the form of a numerical value, a similarity of the particular comparison dataset VDS to the medical dataset PDS. On the basis of this similarity measure, those comparison datasets VDS that have a certain similarity to the medical dataset PDS can then be identified as reference datasets RDS.

In a first step S40-A, a data descriptor is generated on the basis of the medical dataset PDS. Optionally, the generating of the data descriptor can also take into account further information and input parameters, for instance those explained in connection with FIG. 4. The data descriptor can comprise key features of the medical dataset PDS in the form of a feature vector. Since the medical dataset PDS generally has image data and non-image data, the data descriptor can also be based on image features and non-image features. Image features can be extracted using image processing methods. These can comprise the identification, analysis and/or measurement of objects, local and/or global structures, patterns or textures contained in the image data of the medical dataset PDS. The features can further comprise anatomical features and/or structures, for instance the presence of an anatomical landmark or the size, texture or density of an identified organ. In addition, the features can comprise parameters that characterize image values of the image data contained in the medical dataset PDS. For example, these can be parameters that describe a color, a gray level, a contrast, or gradients of these quantities. The features extracted from the non-image data can comprise, for example, metadata on the image data of the medical dataset PDS. In addition, the features can relate to further context data of the patient to be assessed. For example, these features can relate to demographic details about the patient, one or more pre-existing conditions, risk factors, pre-existing diagnoses and findings, laboratory values, vital signs, etc. The data descriptor preferably has a multiplicity of features that in total characterize the medical dataset PDS. In some embodiments, step S40-A is completed using the appropriately designed analysis function AF.

In step S40-B, corresponding data descriptors of the comparison datasets VDS are obtained. The corresponding data descriptors can be generated in a similar way to the data descriptor of the medical dataset PDS and in particular have the same structure as this. According to some embodiments, the corresponding data descriptors have already been generated in advance and are stored with the comparison datasets VDS in the storage facility RD.

In the subsequent step S40-C, a similarity metric is evaluated, which can be used to quantify a similarity between the data descriptor of the medical dataset PDS and the corresponding data descriptors of the comparison datasets VDS. This process can take into account all, or just some, of the comparison datasets VDS contained in the storage unit. In other words, in step S40-C, a similarity measure can thus be obtained for each comparison dataset VDS taken into account. According to some embodiments, the similarity metric can be a distance in vector space between the feature vectors of the medical dataset PDS and the comparison datasets VDS. For example, the distance can be given as a Euclidean distance. According to further examples, the similarity metric can be defined as a cosine similarity between the data descriptors. According to other examples, the similarity of individual features be weighted individually.

The similarity measure can then be used to select from the comparison datasets VDS those datasets that have a certain similarity to the medical dataset PDS. According to exemplary embodiments, all the comparison datasets VDS having a similarity measure above a predetermined threshold can be selected.

One or more, and in particular all the steps S40-A-S40-C, can be executed by the (appropriately designed) analysis function AF. The analysis function AF can comprise one or more trained functions for this purpose. In particular, the extracting of the data descriptors can be carried out with the aid of a trained function.

Different data sources within the medical dataset or the comparison datasets VDS can be processed inside different trained encoder paths. For instance, one encoder path can be designed to extract features from medical images, whereas another encoder path can be designed to extract features from, in particular unstructured, text documents. Likewise, the further information and input parameters mentioned in connection with FIG. 4 can be taken into account. These input parameters can also be translated into features via suitable encoder paths. The totality of the encoder paths can also be referred to as a (trained) encoder segment of the analysis function AF.

In addition, the analysis function can have a trained decoder or classification path, which is designed to output a similarity of feature vectors input to the decoder path. In particular, the decoder path can apply a learned similarity metric to the feature vectors for this purpose. The decoder path can also be referred to as a (trained) classifier segment of the analysis function AF.

Alternatively, a trained function can also be designed to give the similarity measure directly, i.e. without a tangible intermediate step of generating data descriptors.

In addition to trained functions, traditional functions that are not based on artificial intelligence can obviously also be used. An example of these would be what are known as texture classification algorithms.

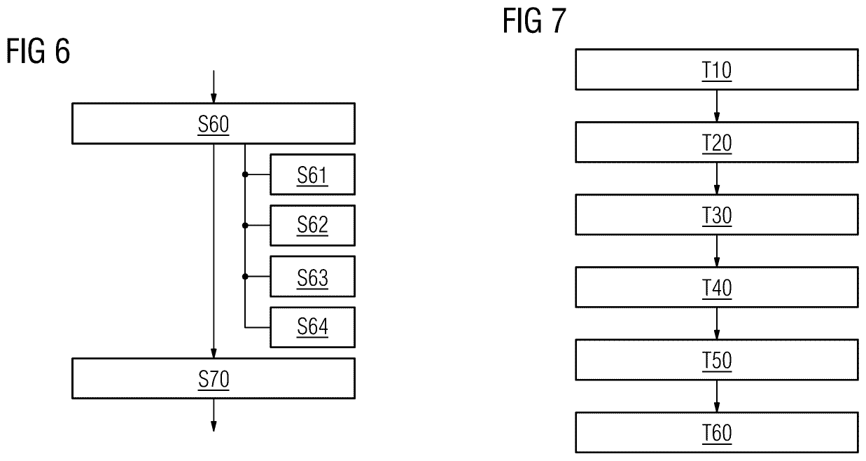

FIG. 6 depicts optional method steps for automated editing and/or populating of the document model structure DVS. The steps shown in FIG. 6 can be executed, for example, as part of providing the document model structure DVS.

In step S61, individual input fields DF of the document model structure DVS can be modified or populated automatically on the basis of already available information. For example, information can be derived for this purpose from the medical dataset PDS, the reference datasets RDS and/or one or more user inputs, and input into associated input fields DF of the document model structure DVS. For instance, measured values of a lesion can be input into a document model structure DVS suitable for an assessment of the lesion.

The steps S62, S63 and S64 are concerned with the automated evaluation and utilization of unstructured information in the reference findings report R-MBB. For instance, text present in the reference datasets RDS can be analyzed, and reused in the providing of the document model structure. This can involve recognizing, for example, those text passages that are specific to the reference dataset RDS and cannot be transferred. These can be, for example, measured values or personal data of the patient. Likewise, more general and thus transferable text passages can be recognized.

For this purpose, in step S62, first can be provided a language analysis algorithm, which is designed to adapt an assessment text to a medical dataset PDS by evaluating the assessment text and, optionally, the medical dataset PDS. Adapting can here comprise in the simplest case removing specific information from the assessment text or replacing it with a placeholder. In more advanced applications, adapting can also comprise replacing specific information in the reference dataset RDS with the corresponding specific information in the medical dataset PDS. The language analysis algorithm can be provided, for example, by being kept available as executable computer code in a memory of the back-end computing facility 20.

In particular, the language analysis algorithm can be a computer linguistics algorithm that is designed to recognize text containing natural language, and assign a meaning to (at least part of) said text. For instance, the computer linguistics algorithm can be designed to recognize certain key words or numerical values in an assessment text, and, if applicable, to substitute these or assign to them a placeholder. In addition, the language analysis algorithm can be designed to search through the medical dataset PDS in a similar manner for corresponding information that can be used to replace the recognized elements.

Numerous applications and functions are generally known from the prior art that can be applied in a language analysis algorithm of this form. Methods of latent semantic analysis can be cited as an example.

In step S63, the assessment text is then adapted by applying the language analysis algorithm to the assessment text. Optionally in this step, the medical dataset PDS can also be input to the language analysis algorithm. An adapted assessment text is thereby obtained that can be used in step S64 to pre-populate the identified document model structure DVS.

FIG. 7 shows an exemplary embodiment of a computer-implemented method for providing a an analysis function AF comprising a trained function. The order of the method steps is limited neither by the sequence shown nor by the chosen numbering. Thus the order of the steps can be interchanged if applicable, and individual steps can be omitted. Moreover, one or more steps, in particular a sequence of steps, and optionally the entire method, can be executed repeatedly.

In step T10, training input data is provided, which training input data has at least one training dataset and a plurality of training comparison datasets, with which the training dataset can be compared by the analysis function AF. The training comparison datasets preferably do not contain the training dataset. Optionally, the training input data can also have further information for ascertaining reference datasets, for instance the user inputs described herein, notes on medical abnormalities, or their variations over time.

In step T20, training output data is provided. The training output data indicates at least one verified reference dataset within the training comparison datasets that has a certain similarity to the training dataset. The verified reference dataset can have been specified by a human expert or otherwise determined by selection rules.

In a step T30, the analysis function AF is applied to the training input data in order to generate intermediate output data. The intermediate output data corresponds here to a prediction of at least one reference dataset by the analysis function. The analysis function AF can already be pre-trained, i.e. one or more parameters of the analysis function AF have already been adapted by the described training method and/or another training method. Alternatively, the one or more parameters of the analysis function AF may not yet have been adapted by the training data, in particular the one or more parameters may be preset with a constant value and/or with a random value.

In step T40, the intermediate output data is compared with the training output data, whereupon, in step T50, the trained function TF is adapted on the basis of the comparison. This can be done, for example, on the basis of a cost functional, which penalizes reference datasets that are incorrectly selected. In particular, an external classifier can be used for this purpose, which rates the similarity of the intermediate output data to the training output data, and the less similar the intermediate output data and the training output data, the more it penalizes the analysis function AF. Using an external classifier to determine the cost functional is advantageous in particular when the reference datasets have a similar structure amongst themselves that differs from that of the medical dataset to be processed later. This can be the case, for example, when the comparison datasets VDS contain largely findings reports R-MBB whereas the medical data PDS comprises further information. By using an external classifier, training output data can be generated more easily, and feedback of better resolution can be given to the analysis function AF during the training, which can improve the training.

Then, in step T50, one or more parameters of the analysis function AF can be adapted in particular such that the cost functional is minimized, for instance via backpropagation. In order to minimize the cost functional, the comparison is carried out for different paired sets composed of training output data and training output data and also intermediate output data until a local minimum of the cost functional is reached and the analysis function AF is working satisfactorily. In step T60, the trained function TF adapted in this way is finally provided.

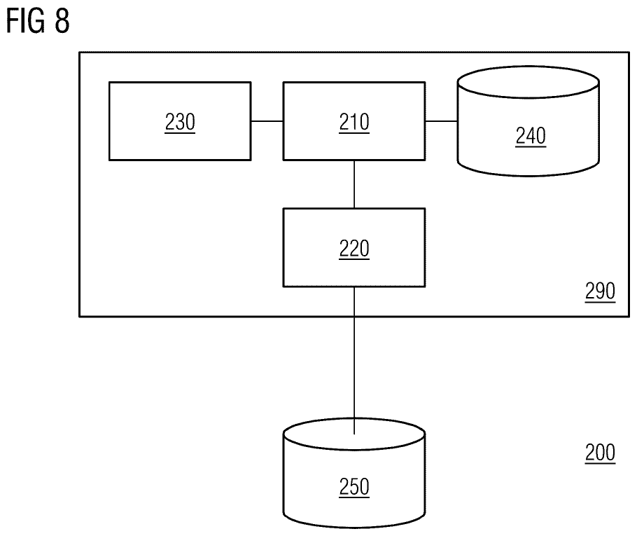

FIG. 8 shows an exemplary embodiment of a system 200 for training an analysis function AF comprising one or more trained functions. The system comprises a processor 210, an interface 220, a main memory 230, a local storage medium 240 and a database 250. The processor 210, the interface 220, the main memory 230 and the local storage medium 240 can be embodied as a computer 290. The processor 210 controls the computer 290 by executing computer program instructions. The computer program instructions can be stored in the main memory 230 or in the local storage medium 240 and loaded into the main memory 230 when the execution of the computer program instructions is required. The method steps shown in FIG. 7 can be defined by the computer program instructions stored in the main memory 230 and/or local storage medium 240.

The database 250 is a storage facility such as a Cloud or a local storage medium, which serves as an archive for the training datasets introduced above. The database 250 can be connected to the computer 290 via a wireless or wired connection. It is also possible to implement the database 250 and the computer 290 as a single device. The interface 220 is designed in particular to establish a data connection between the computer 290 and the database 250.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

While exemplary embodiments have been described in detail in particular with reference to the figures, it should be mentioned that numerous variations are possible. It should also be mentioned that the exemplary embodiments are merely examples that shall not restrict the scope of protection, the usage or the construction in any way. Instead, the description above provides a person skilled in the art with guidance for implementing at least one exemplary embodiment, where various modifications, in particular alternative or additional features and/or modification of the function and/or arrangement of the described constituent parts, can be made as required by a person skilled in the art without departing from the subject matter defined in each of the accompanying claims or from its legal equivalent and/or without departing from their scope of protection.

What is claimed is:

1. A computer-implemented method for providing a document model structure for producing a medical findings report in an assessment of a patient to be assessed, the computer-implemented method comprising:
receiving, at a computing system, a medical dataset of the patient to be assessed;
providing, at the computing system, a plurality of comparison datasets, the plurality of comparison datasets differing from the medical dataset, wherein each of the plurality of comparison datasets has at least one reference medical findings report;
providing an analysis function configured to ascertain, for a medical dataset, at least one reference dataset from the plurality of comparison datasets;
ascertaining, by the computing system, the at least one reference dataset from the plurality of comparison datasets by applying the analysis function to the medical dataset and the plurality of comparison datasets;
identifying, by the computing system, at least one document model structure for the patient based on the at least one reference medical findings report associated with the at least one reference dataset; and
providing, by the computing system, the at least one document model structure for producing a medical findings report for the patient to be assessed.

2. The computer-implemented method as claimed in claim 1, further comprising:
receiving, at the computing system, input directed to the medical dataset, wherein
the analysis function is further configured to ascertain the at least one reference dataset additionally based on the input directed to the medical dataset, and
the ascertaining ascertains the at least one reference dataset by applying the analysis function to the input.

3. The computer-implemented method as claimed in claim 2, wherein the input is directed to one or more of the following inputs:
defining a region of interest in the medical dataset;
detecting a medical abnormality in the medical dataset;
producing a measured value of an abnormality exhibited in the medical dataset;
selecting an analysis tool for producing a measured value of an abnormality exhibited in the medical dataset; or
setting one or more reproduction parameters for presenting the medical dataset in a user interface.

4. The computer-implemented method as claimed in claim 1, further comprising:
providing a detection function configured to detect medical abnormalities in the medical dataset in an automated manner, the detection function being different from the analysis function; and
applying, by the computing system, the detection function to the medical dataset to detect at least one medical abnormality and provide information about the at least one medical abnormality, wherein
the analysis function is further configured to ascertain the at least one reference dataset additionally based on information about at least one medical abnormality detected in the medical dataset, and
the ascertaining ascertains the at least one reference dataset additionally by applying the analysis function to the information about the at least one medical abnormality.

5. The computer-implemented method as claimed in claim 1, further comprising:
obtaining, by the computing system, a medical abnormality in the medical dataset;
determining, by the computing system, a development over time of the medical abnormality based on at least one of the medical dataset or further medical information on the patient to be assessed, the further medical information being different from the medical dataset, wherein
the analysis function is further configured to ascertain the at least one reference dataset additionally based on the development over time of the medical abnormality, and
the ascertaining ascertains the at least one reference dataset additionally by applying the analysis function to the development over time.

6. The computer-implemented method as claimed in claim 1, wherein
the reference medical findings reports associated with the plurality of comparison datasets are each based on at least one standardized template component for producing a medical findings report,
the identifying includes identifying at least one template component based on the at least one reference medical findings report associated with the at least one reference dataset, and
the providing the at least one document model structure includes providing the at least one template component as a document model structure.

7. The computer-implemented method as claimed in claim 1, further comprising:
receiving, by the computing system, input directed to editing of the at least one document model structure;
producing, by the computing system, a medical findings report based on the at least one document model structure and the input directed to the editing; and
providing, by the computing system, the medical findings report.

8. The computer-implemented method as claimed in claim 1, wherein
the at least one document model structure is populated with an assessment text containing natural language; and
the computer-implemented method further includes providing a language analysis algorithm, which is configured to adapt the assessment text to the medical dataset by evaluating the assessment text and the medical dataset,
adapting, by the computing system, the assessment text to the medical dataset by applying the language analysis algorithm to the assessment text and the medical dataset, and
pre-populating, by the computing system, the at least one document model structure with the adapted assessment text, wherein
the providing provides the pre-populated at least one document model structure.

9. The computer-implemented method as claimed in claim 1, wherein
the analysis function is configured to calculate a similarity measure between the medical dataset and a comparison dataset, the similarity measure indicating a similarity between the medical dataset and the comparison dataset, and
the ascertaining the at least one reference dataset includes calculating a similarity measure for each of the plurality of comparison datasets by applying the analysis function to the medical dataset and the plurality of comparison datasets, and
the at least one reference dataset is ascertained from the plurality of comparison datasets based on the similarity measures.

10. The computer-implemented method as claimed in claim 9, wherein
the analysis function is configured to
extract at least one of a data descriptor from the medical dataset or a corresponding data descriptor from the comparison dataset, the data descriptor stating attributes of an underlying medical dataset that are relevant to ascertaining reference datasets, and the corresponding data descriptor stating attributes of an underlying medical dataset that is relevant to ascertaining the reference datasets, and
calculate a similarity measure between the medical dataset and the comparison dataset based on associated data descriptors by inputting the associated data descriptors into a similarity metric, wherein
the similarity measure for the comparison dataset is calculated by
obtaining a data descriptor from the medical dataset by applying the analysis function to the medical dataset,
obtaining a corresponding data descriptor for the comparison dataset by applying the analysis function to the comparison dataset, and
calculating the similarity measure for the comparison dataset based on the data descriptor and the corresponding data descriptor by applying the analysis function to the data descriptor and the corresponding data descriptor.

11. The computer-implemented method as claimed in claim 1, wherein the analysis function comprises a trained function.

12. The computer-implemented method as claimed in claim 1, wherein
the identifying of the at least one document model structure includes identifying a plurality of document model structures for selection by a user,
the providing provides the plurality of document model structures to the user via a user interface,
the computer-implemented method further includes receiving, via the user interface, input by the user selecting the at least one document model structure, and
the providing provides the at least one document model structure selected by the user in the user interface for further revision by the user.

13. A system for providing a document model structure for producing a medical findings report for a patient to be assessed, the system comprising:
an interface configured to receive a medical dataset of the patient to be assessed, and to provide a plurality of comparison datasets, each of the plurality of comparison datasets having at least one reference medical findings report, and the plurality of comparison datasets being different from the medical dataset; and
a computing system configured to
host an analysis function configured to ascertain, for the medical dataset, at least one reference dataset from the plurality of comparison datasets,
ascertain the at least one reference dataset from the plurality of comparison datasets by applying the analysis function to the medical dataset and the plurality of comparison datasets,
identify at least one document model structure for the patient based on the at least one reference medical findings report associated with the at least one reference dataset, and
provide the at least one document model structure via the interface.

14. A non-transitory computer program product including a program loadable directly into a memory of a programmable computing system, the program including program instructions to perform the method as claimed in claim 1 when the program is executed at the computing system.

15. A non-transitory computer-readable storage medium storing computer-executable program segments that, when executed at a computing system, cause the computing system to perform the method as claimed in claim 1.

16. The computer-implemented method as claimed in claim 2, wherein
the reference medical findings reports associated with the plurality of comparison datasets are each based on at least one standardized template component for producing a medical findings report,
the identifying includes identifying at least one template component based on the at least one reference medical findings report associated with the at least one reference dataset, and
the providing the at least one document model structure includes providing the at least one template component as a document model structure.

17. The computer-implemented method as claimed in claim 2, further comprising:
providing a detection function configured to detect medical abnormalities in the medical dataset in an automated manner, the detection function being different from the analysis function; and
applying, by the computing system, the detection function to the medical dataset to detect at least one medical abnormality and provide information about the at least one medical abnormality, wherein
the analysis function is further configured to ascertain the at least one reference dataset additionally based on information about at least one medical abnormality detected in the medical dataset, and
the ascertaining ascertains the at least one reference dataset additionally by applying the analysis function to the information about the at least one medical abnormality.

18. The computer-implemented method as claimed in claim 2, further comprising:
receiving, by the computing system, input directed to editing of the at least one document model structure;
producing, by the computing system, a medical findings report based on the at least one document model structure and the input directed to the editing; and
providing, by the computing system, the medical findings report.

19. The computer-implemented method as claimed in claim 4, further comprising:
obtaining, by the computing system, the at least one medical abnormality in the medical dataset;
determining, by the computing system, a development over time of the at least one medical abnormality based on at least one of the medical dataset or further medical information on the patient to be assessed, the further medical information being different from the medical dataset, wherein
the analysis function is further configured to ascertain the at least one reference dataset additionally based on the development over time of the at least one medical abnormality, and
the ascertaining ascertains the at least one reference dataset additionally by applying the analysis function to the development over time.

20. The computer-implemented method as claimed in claim 5, wherein
the reference medical findings reports associated with the plurality of comparison datasets are each based on at least one standardized template component for producing a medical findings report,
the identifying includes identifying at least one template component based on the at least one reference medical findings report associated with the at least one reference dataset, and
the providing the at least one document model structure includes providing the at least one template component as a document model structure.
